(19) United States
(12) Patent Application Publication
(10) Pub. No.: US 2020/0245978 A1
(43) Pub. Date: Aug. 6, 2020

(54) FAILURE DIAGNOSIS SYSTEM OF ULTRASONIC ENDOSCOPE APPARATUS, FAILURE DIAGNOSIS METHOD OF ULTRASONIC ENDOSCOPE APPARATUS, AND FAILURE DIAGNOSIS PROGRAM OF ULTRASONIC ENDOSCOPE APPARATUS

(71) Applicant: FUJIFILM Corporation, Tokyo (JP)

(72) Inventor: Kenichi SHIDARA, Kanagawa (JP)

(73) Assignee: FUJIFILM Corporation, Tokyo (JP)

(21) Appl. No.: 16/716,843

(22) Filed: Dec. 17, 2019

(30) Foreign Application Priority Data
Jan. 31, 2019 (JP) 2019-016086

(51) Int. Cl. A61B 8/00; A61B 8/12

(52) U.S. Cl. CPC A61B 8/58; A61B 8/54; A61B 8/12

(57) ABSTRACT

Provided are a failure diagnosis system of an ultrasonic endoscope apparatus, a failure diagnosis method of the ultrasonic endoscope apparatus, and a non-transitory computer readable recording medium storing a failure diagnosis program of the ultrasonic endoscope apparatus capable of performing failure diagnosis of the ultrasonic endoscope apparatus with high accuracy. The system controller acquires a reception signal of an ultrasonic vibrator in a state where ultrasonic waves are not transmitted from the ultrasonic vibrator of an ultrasonic endoscope, and performs failure diagnosis of an ultrasonic endoscope apparatus including the ultrasonic endoscope on the basis of the reception signal.

### CROSS-REFERENCE TO RELATED APPLICATIONS

The present application claims priority under 35 U.S.C. § 119 to Japanese Patent Application No. 2019-016086, filed on Jan. 31, 2019. Each of the above application(s) is hereby expressly incorporated by reference, in its entirety, into the present application.

### BACKGROUND OF THE INVENTION

### 1. Field of the Invention

The present invention relates to a failure diagnosis system of an ultrasonic endoscope apparatus, a failure diagnosis method of the ultrasonic endoscope apparatus, and a non-transitory computer readable recording medium storing a failure diagnosis program of the ultrasonic endoscope apparatus.

### 2. Description of the Related Art

An ultrasonic diagnosis apparatus that respectively drives a plurality of ultrasonic vibrators inside a subject (for example, a patient's body) and transmits and receives ultrasonic waves to acquire an ultrasound image inside the subject is already known (for example, see JP2009-285175A and JP1994-269452A (JP-H06-269452A)). JP2009-285175A and JP1994-269452A (JP-H06-269452A) disclose such an ultrasonic endoscope apparatus. The apparatus disclosed in JP2009-285175A and JP1994-269452A (JP-H06-269452A) performs abnormality detection such as disconnection in an ultrasonic endoscope on the basis of a reception signal of an ultrasonic vibrator in a case where ultrasonic waves are transmitted from the ultrasonic vibrator.

### SUMMARY OF THE INVENTION

The ultrasonic endoscope apparatus includes an ultrasonic endoscope and a main body to which the ultrasonic endoscope is connected. As disclosed in JP2009-285175A and JP1994-269452A (JP-H06-269452A), it is possible to detect an abnormality of the ultrasonic endoscope by transmitting ultrasonic waves from an ultrasonic vibrator and analyzing a reception signal of reflected waves thereof. Since the ultrasonic endoscope uses a minute signal for generating an ultrasound image, the ultrasonic endoscope is easily affected by noise. There are various causes of noise mixture in the minute signal, such as an ultrasonic endoscope itself, or a device included in the main body. The abnormality detection method disclosed in JP2009-285175A and JP1994-269452A (JP-H06-269452A) is to analyze a reception signal obtained in a state where the ultrasonic vibrator is operated in the same manner as in a normal inspection to detect an abnormality. Accordingly, the noise included in the reception signal is buried in the level of the reflected waves of the ultrasonic waves, and thus, it is not possible to determine what kind of noise is generated.

The invention has been made in consideration of the above-mentioned problems, and an object of the invention is to provide a failure diagnosis system of an ultrasonic endoscope apparatus, a failure diagnosis method of the ultrasonic endoscope apparatus, and a non-transitory computer readable recording medium storing a failure diagnosis program of the ultrasonic endoscope apparatus capable of performing failure diagnosis of the ultrasonic endoscope apparatus with high accuracy.

According to an aspect of the invention, there is provided a failure diagnosis system of an ultrasonic endoscope apparatus comprising a failure diagnosis unit that acquires a reception signal of an ultrasonic vibrator of an ultrasonic endoscope in a state where ultrasonic waves are not transmitted from the ultrasonic vibrator and performs failure diagnosis of the ultrasonic endoscope apparatus including the ultrasonic endoscope on the basis of the reception signal.

According to another aspect of the invention, there is provided a failure diagnosis method of an ultrasonic endoscope apparatus comprising: acquiring a reception signal of an ultrasonic vibrator of an ultrasonic endoscope in a state where ultrasonic waves are not transmitted from the ultrasonic vibrator; and performing failure diagnosis of the ultrasonic endoscope apparatus including the ultrasonic endoscope on the basis of the reception signal.

According to still another aspect of the invention, there is provided a non-transitory computer readable recording medium storing a failure diagnosis program of an ultrasonic endoscope apparatus for causing a computer to execute: a step of acquiring a reception signal of an ultrasonic vibrator of an ultrasonic endoscope in a state where ultrasonic waves are not transmitted from the ultrasonic vibrator; and a step of performing failure diagnosis of the ultrasonic endoscope apparatus including the ultrasonic endoscope on the basis of the reception signal.

According to the invention, it is possible to provide a failure diagnosis system of an ultrasonic endoscope apparatus, a failure diagnosis method of the ultrasonic endoscope apparatus, and a non-transitory computer readable recording medium storing a failure diagnosis program of the ultrasonic endoscope apparatus capable of performing failure diagnosis of the ultrasonic endoscope apparatus with high accuracy.

### BRIEF DESCRIPTION OF THE DRAWINGS

FIG. 1 is a diagram showing a schematic configuration of an ultrasonic endoscope apparatus 10.

FIG. 2 is an enlarged plan view showing a distal end part of an insertion part 22 of an ultrasonic endoscope 12 and the vicinity thereof.

FIG. 3 is a diagram showing a cross section of a distal end part 40 of the insertion part 22 of the ultrasonic endoscope 12, taken along a section I-I shown in FIG. 2.

FIG. 4 is a block diagram showing a configuration of the ultrasonic endoscope 12 and an ultrasonic processor device 14.

FIG. 5 is a diagram showing functional blocks of a system controller 152.

FIG. 6 is a diagram showing an example of a reception signal acquired in a case where ultrasonic waves are not transmitted.

FIG. 7 is a diagram showing an example in which noise is mixed in a reception signal acquired in a case where ultrasonic waves are not transmitted.

FIG. 8 is a diagram showing an example of a reception signal acquired in a case where ultrasonic waves are not transmitted.

### DESCRIPTION OF THE PREFERRED EMBODIMENTS

Overview of Ultrasonic Diagnosis Apparatus

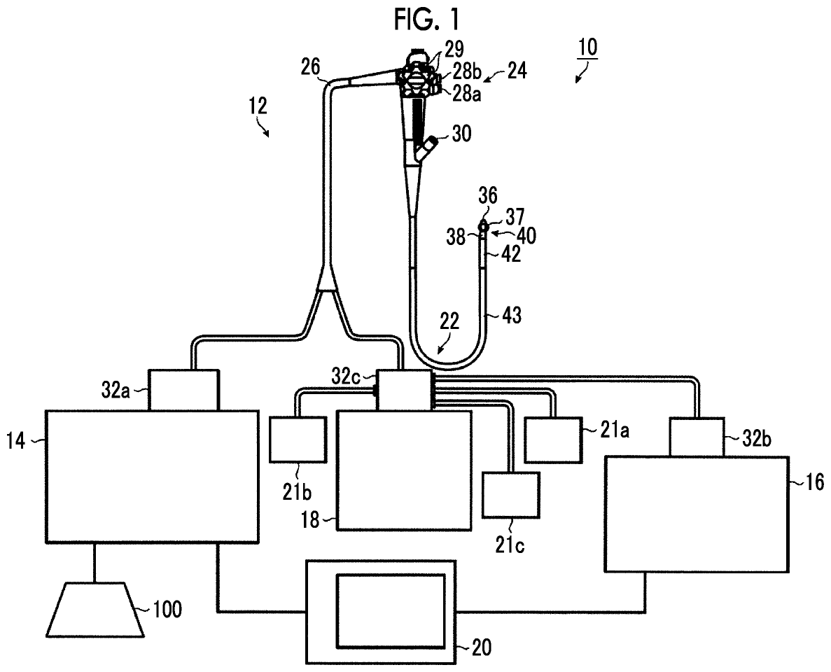

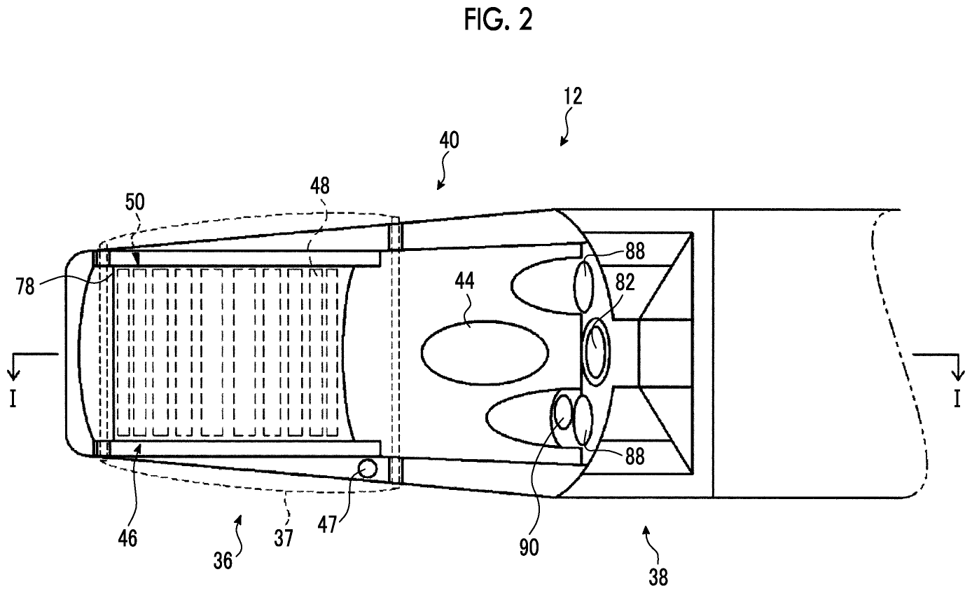

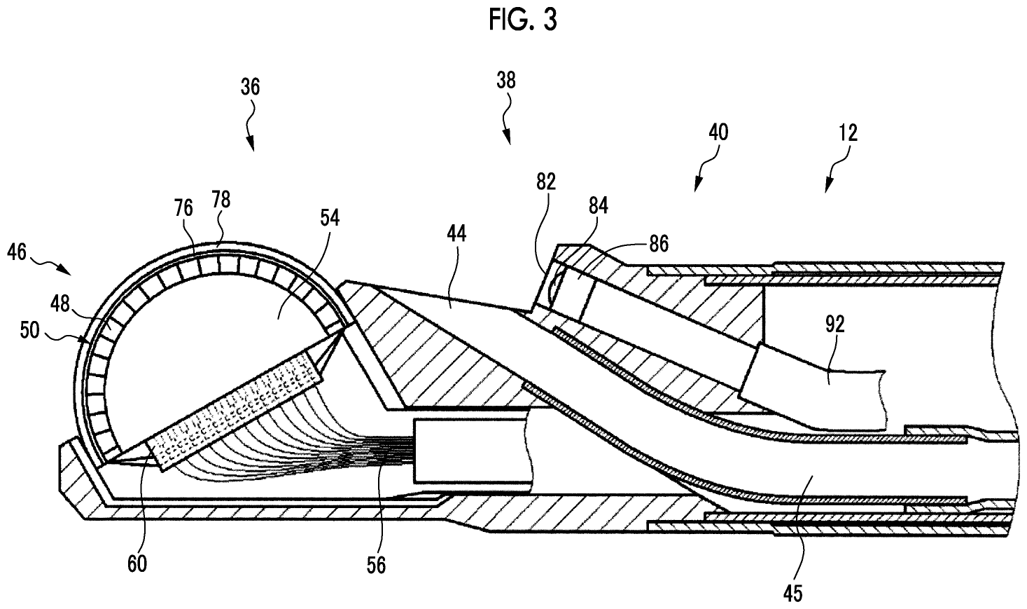

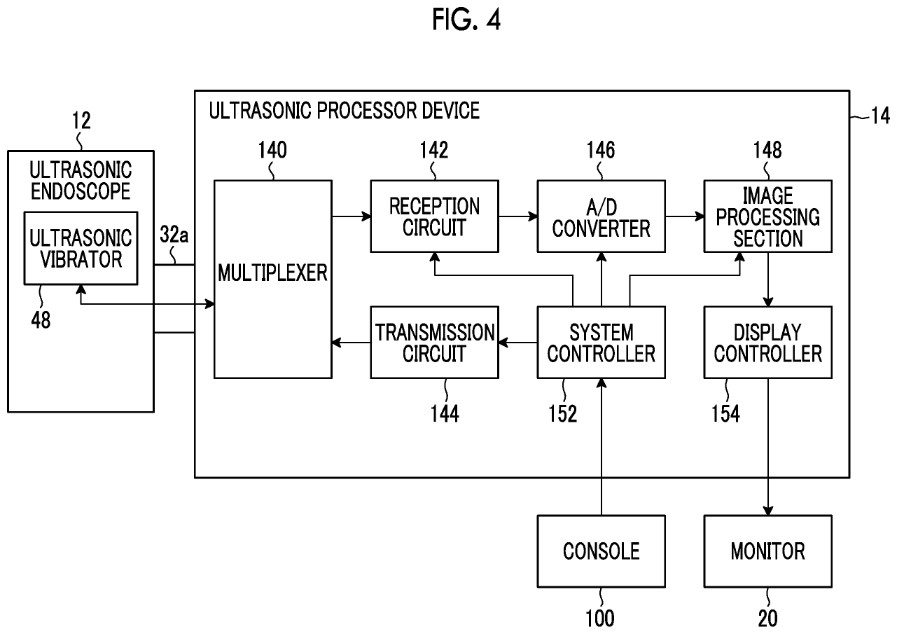

An outline of an ultrasonic endoscope apparatus 10 including a failure prediction system according to an embodiment of the invention will be described with reference to FIGS. 1 to 4. FIG. 1 is a diagram showing a schematic configuration of the ultrasonic endoscope apparatus 10. FIG. 2 is an enlarged plan view of a distal end part of an insertion part 22 of an ultrasonic endoscope 12 and the vicinity thereof. In FIG. 2, for ease of illustration, a balloon 37 to be described later is shown by a broken line. FIG. 3 is a diagram showing a cross section of a distal end part 40 of the insertion part 22 of the ultrasonic endoscope 12, taken along a section I-I shown in FIG. 2. FIG. 4 is a block diagram showing a configuration of the ultrasonic endoscope 12 and the ultrasonic processor device 14.

The ultrasonic endoscope apparatus 10 is used for observing a state of an observation target portion in the body of a patient that is a subject using ultrasonic waves (hereinafter, referred to as ultrasonic diagnosis). Here, the observation target portion is a portion that is difficult to inspect from a body surface (outside) of the patient, which is the gallbladder or pancreas, for example. By using the ultrasonic endoscope apparatus 10, a state of the observation target portion and the presence or absence of an abnormality thereof may be ultrasonically diagnosed through the digestive tract such as the esophagus, stomach, duodenum, small intestine, and large intestine that are body cavities of the patient.

As shown in FIG. 1, the ultrasonic endoscope apparatus 10 includes the ultrasonic endoscope 12, an ultrasonic processor device 14, an endoscope processor device 16, a light source device 18, a monitor 20, and a console 100. Further, as shown in FIG. 1, a water supply tank 21a, a suction pump 21b, and an air supply pump 21c are provided as accessory devices of the ultrasonic endoscope apparatus 10. Further, a pipeline (not shown) that serves as a flow path for water and gas is formed in the ultrasonic endoscope 12. The ultrasonic processor device 14, the endoscope processor device 16, and the light source device 18 configure a main body of the ultrasonic endoscope apparatus 10.

As shown in FIG. 1, the ultrasonic endoscope 12 includes an insertion part 22 that is inserted into a body cavity of a patient, and an operation part 24 that is operated by an operator (user) such as a doctor or a technician. Further, as shown in FIGS. 2 and 3, an ultrasonic vibrator unit 46 including a plurality of ultrasonic vibrators 48 is attached to a distal end part 40 of the insertion part 22.

With the function of the ultrasonic endoscope 12, the operator may acquire an endoscope image of an inner wall of the body cavity of the patient and an ultrasound image of the observation target portion. The endoscope image is an image obtained by imaging the inner wall of the body cavity of the patient using an optical technique. The ultrasound image is an image obtained by receiving reflected waves (echoes) of ultrasonic waves transmitted from the body cavity of the patient toward the observation target portion and imaging a reception signal thereof.

The ultrasonic processor device 14 is connected to the ultrasonic endoscope 12 through a universal cord 26 and an ultrasound connector 32a provided at an end part thereof, as shown in FIG. 1. The ultrasonic processor device 14 controls the ultrasonic vibrator unit 46 of the ultrasonic endoscope 12 to transmit ultrasonic waves to the ultrasonic vibrator unit 46. Further, the ultrasonic processor device 14 images a reception signal in a case where the ultrasonic vibrator unit 46 receives reflected waves (echoes) of ultrasonic waves to generate an ultrasound image.

As shown in FIG. 1, the endoscope processor device 16 is connected to the ultrasonic endoscope 12 through the universal cord 26 and an endoscope connector 32b provided at an end part of the universal cord 26. The endoscope processor device 16 acquires image data of an observation target adjacent portion imaged by the ultrasonic endoscope 12 (specifically, an imaging element 86 to be described later), and performs predetermined image processing with respect to the acquired image data to generate an endoscope image. The observation target adjacent portion is a portion of the inner wall of the body cavity of the patient, which is adjacent to the observation target portion.

As shown in FIG. 1, the light source device 18 is connected to the ultrasonic endoscope 12 through the universal cord 26 and a light source connector 32c provided at the end part thereof. The light source device 18 emits white light, formed of three primary colors of red light, green light and blue light, or specific wavelength light in imaging the observation target adjacent portion using the ultrasonic endoscope 12. The light emitted from the light source device 18 propagates in the ultrasonic endoscope 12 through a light guide (not shown) included in the universal cord 26, and then, is emitted from the ultrasonic endoscope 12 (specifically, an illumination window 88 to be described later). Thus, the observation target adjacent portion is illuminated by the light from the light source device 18.

In this embodiment, the ultrasonic processor device 14 and the endoscope processor device 16 are configured by two devices (computers) that are separately provided. However, the invention is not limited to this configuration, and both the ultrasonic processor device 14 and the endoscope processor device 16 may be configured by a single device.

As shown in FIG. 1, the monitor 20 is connected to the ultrasonic processor device 14 and the endoscope processor device 16, and displays an ultrasound image generated by the ultrasonic processor device 14 and an endoscope image generated by the endoscope processor device 16. Regarding the display of the ultrasound image and the endoscope image, either one of the images may be switched and displayed on the monitor 20, or both the images may be simultaneously displayed. Further, a configuration in which the display methods are able to be discretionally selected or changed may be used.

In this embodiment, the ultrasound image and the endoscope image are displayed on one monitor 20, but an ultrasound image display monitor and an endoscope image display monitor may be separately provided. Further, a display form other than the monitor 20 may be used. For example, a form in which an ultrasound image and an endoscope image are displayed on a display of a personal terminal carried by an operator may be used.

The console 100 is an input device provided for an operator to input information necessary for ultrasonic diagnosis or for an operator to instruct the ultrasonic processor device 14 to start the ultrasonic diagnosis. The console 100 includes, for example, a keyboard, a mouse, a trackball, a touch pad, a touch panel, and the like, and is connected to a system controller 152 of the ultrasonic processor device 14 as shown in FIG. 4. In a case where the console 100 is operated, the system controller 152 of the ultrasonic processor device 14 controls each part of the device (for example, a reception circuit 142 and a transmission circuit 144 to be described later) according to the operation content.

The ultrasonic endoscope apparatus 10 configured as described above performs initialization for activation in a case where electric power is supplied. In a case where the ultrasonic endoscope 12 is connected to the main body at the same time as the electric power is supplied, the system controller 152 of the ultrasonic processor device 14 operates the ultrasonic endoscope 12 after the initialization to proceed to a live mode. The live mode is a mode for sequentially displaying (real-time display) ultrasound images (motion pictures) obtained at a predetermined frame rate. In a case where the ultrasonic endoscope 12 is not connected to the main body at a time point when the electric power is supplied, the system controller 152 of the ultrasonic processor device 14 operates the ultrasonic endoscope 12 at a time point when the ultrasonic endoscope 12 is connected thereto after the initialization to proceed to the live mode. In a state where the ultrasonic endoscope 12 is connected to the main body, it is possible to start the live mode at an unspecified timing (for example, a timing for starting inspection of a subject (a timing immediately before the ultrasonic endoscope 12 is inserted into the body cavity)) by operating the console 100.

In the ultrasonic endoscope apparatus 10, at an unspecified timing in a period during which the ultrasonic endoscope 12 is not inserted into the body cavity in a state where the ultrasonic endoscope 12 is connected to the main body (in other words, in a period during which the ultrasonic endoscope 12 is not used), the ultrasonic processor device 14 performs a failure diagnosis process for diagnosing a failure of the ultrasonic endoscope apparatus 10. The failure diagnosis process will be described later.

The period during which the ultrasonic endoscope 12 is not used may be determined as follows, for example. 1) A period until an inspection starting instruction is performed by operating the console 100 after electric power is supplied is determined as the period during which the ultrasonic endoscope 12 is not used. 2) A period during which a change in an endoscope image acquired from the ultrasonic endoscope 12 is small after electric power is supplied is determined as the period during which the ultrasonic endoscope 12 is not used. 3) A motion sensor such as an acceleration sensor is provided in the ultrasonic endoscope 12, and a period during which the amount of motion of the ultrasonic endoscope 12 is smaller than a predetermined value is determined as the period during which the ultrasonic endoscope 12 is not used. 4) A maintenance mode is provided in the ultrasonic endoscope apparatus 10, and a period during which the ultrasonic endoscope apparatus 10 is set to the maintenance mode is determined as the period during which the ultrasonic endoscope 12 is not used.

Configuration of Ultrasonic Endoscope

Next, a configuration of the ultrasonic endoscope 12 will be described with reference to FIGS. 1 to 4. The ultrasonic endoscope 12 includes the insertion part 22 and the operation part 24 as shown in FIG. 1. As shown in FIG. 1, the insertion part 22 includes the distal end part 40, a bending part 42, and a flexible part 43 in order from the distal end side (free end side). As shown in FIG. 2, the distal end part 40 is provided with an ultrasound observation part 36 and an endoscope observation part 38.

Further, as shown in FIGS. 2 and 3, the distal end part 40 is provided with a treatment instrument outlet 44. The treatment instrument outlet 44 serves as an outlet of a treatment instrument (not shown) such as a pair of forceps, a puncture needle, or a high-frequency knife, and also serves as a suction port for sucking a sucked substance such as blood and filth in the body.

Further, as shown in FIG. 2, a cleaning nozzle 90 formed to clean surfaces of an observation window 82 and an illumination window 88 is provided at the distal end part 40. Air or cleaning liquid is ejected from the cleaning nozzle 90 toward the observation window 82 and the illumination window 88.

Further, as shown in FIGS. 1 and 2, a balloon 37 that is able to be inflated and deflated is attached to the distal end part 40 at a position where the ultrasonic vibrator unit 46 is covered. The balloon 37 is disposed in the body cavity of the patient together with the ultrasonic vibrator unit 46. Then, water (specifically, de-aired water) as an ultrasonic transmission medium is injected into the balloon 37 from a water supply port 47 formed in the vicinity of the ultrasonic vibrator unit 46 at the distal end part 40, and thus, the balloon 37 is inflated. In a case where the inflated balloon 37 comes into contact with the inner wall of the body cavity (for example, around the observation target adjacent portion), air is excluded from between the ultrasonic vibrator unit 46 and the inner wall of the body cavity. Thus, it is possible to prevent attenuation of ultrasonic waves and their reflected waves (echoes) in the air.

As shown in FIG. 1, the bending part 42 is a part provided on a proximal end side (a side opposite to the side where the ultrasonic vibrator unit 46 is provided) with reference to the distal end part 40 in the insertion part 22, which is able to be freely bent. As shown in FIG. 1, the flexible part 43 is a part that connects the bending part 42 and the operation part 24, has flexibility, and is provided in an elongated state.

As shown in FIG. 1, the operation part 24 is provided with a pair of angle knobs 29 and a treatment instrument insertion port 30. In a case where each angle knob 29 is rotated, the bending part 42 is remotely operated to be bent and deformed. By this deformation operation, the distal end part 40 of the insertion part 22 provided with the ultrasound observation part 36 and the endoscope observation part 38 may be directed in a desired direction. The treatment instrument insertion port 30 is a hole formed for insertion of a treatment instrument such as a pair of forceps, and communicates with the treatment instrument outlet 44 through a treatment instrument channel 45 (see FIG. 3).

As shown in FIG. 1, the operation part 24 is provided with an air/water supply button 28a for opening or closing an air/water supply pipeline (not shown) that extends from a water supply tank 21a, and a suction button 28b for opening or closing a suction line (not shown) that extends from a suction pump 21b. A gas such as air sent from an air supply pump 21c and water in the water supply tank 21a flow through the air/water supply pipeline. In a case where the air/water supply button 28a is operated, a part to be opened of the air/water supply pipeline is switched, and gas and water ejecting outlets are also switched in a corresponding form between the cleaning nozzle 90 and the water supply port 47. That is, through the operation of the air/water supply button 28a, the cleaning of the endoscope observation part 38 and the inflation of the balloon 37 may be selectively performed.

The suction line is provided for sucking a sucked substance in the body cavity sucked from the cleaning nozzle 90 or for sucking the water in the balloon 37 through the water supply port 47. In a case where the suction button 28b is operated, a portion to be opened of the suction line is switched, and the suction port is also switched in a corresponding form between the cleaning nozzle 90 and the water supply port 47. That is, an object sucked by the suction pump 21b may be switched through the operation of the suction button 28b.

As shown in FIG. 1, at the other end of the universal cord 26, the ultrasound connector 32a connected to the ultrasonic processor device 14, the endoscope connector 32b connected to the endoscope processor device 16, and the light source connector 32c connected to the light source device 18 are provided. The ultrasonic endoscope 12 is detachably connected to the ultrasonic processor device 14, the endoscope processor device 16, and the light source device 18 through the connectors 32a, 32b, and 32c, respectively.

Next, among the components of the ultrasonic endoscope 12, the ultrasound observation part 36 and the endoscope observation part 38 will be described in detail.

Ultrasound Observation Part

The ultrasound observation part 36 is a part provided for acquiring an ultrasound image, and is disposed on the distal end side in the distal end part 40 of the insertion part 22 as shown in FIGS. 2 and 3. As shown in FIG. 3, the ultrasound observation part 36 includes the ultrasonic vibrator unit 46, a plurality of coaxial cables 56, and a flexible printed circuit (FPC) 60.

As shown in FIG. 3, the ultrasonic vibrator unit 46 is a convex probe in which a plurality of ultrasonic vibrators 48 are arranged in an arc shape, and transmits ultrasonic waves in a radial shape (arc shape). However, the type (model) of the ultrasonic vibrator unit 46 is not particularly limited, and may be any other type that can transmit and receive ultrasonic waves, for example, a sector type, a linear type, a radial type, and the like.

As shown in FIG. 3, the ultrasonic vibrator unit 46 is configured by laminating a backing material layer 54, an ultrasonic vibrator array 50, an acoustic matching layer 76, and an acoustic lens 78.

As shown in FIG. 3, the ultrasonic vibrator array 50 is configured of a plurality of ultrasonic vibrators 48 (ultrasonic transducers) that are arranged in a one-dimensional array shape. More specifically, the ultrasonic vibrator array 50 has a configuration in which N (for example, N=128) ultrasonic vibrators 48 are arranged in a convexly curved shape along an axial direction of the distal end part 40 (longitudinal axis direction of the insertion part 22) at equal intervals. The ultrasonic vibrator array 50 may have a configuration in which the plurality of ultrasonic vibrators 48 are arranged in a two-dimensional array shape.

Each of the N ultrasonic vibrators 48 is configured by disposing electrodes on both surfaces of a single crystal vibrator that is a piezoelectric element. As the single crystal vibrator, any one of quartz, lithium niobate, lead magnesium niobate (PMN), lead zinc niobate (PZN), lead indium niobate (PIN), lead titanate (PT), lithium tantalate, langasite, or zinc oxide may be used. The electrodes include individual electrodes (not shown) that are individually provided for each of the plurality of ultrasonic vibrators 48 and a ground electrode (not shown) common to the plurality of ultrasonic vibrators 48. Further, the electrodes are electrically connected to the ultrasonic processor device 14 through the coaxial cable 56 and the FPC 60.

Each ultrasonic vibrator 48 is supplied with a pulsed drive voltage as an input signal from the ultrasonic processor device 14 through the coaxial cable 56. In a case where the drive voltage is applied to the electrodes of the ultrasonic vibrator 48, the piezoelectric element expands and contracts, so that the ultrasonic vibrator 48 is driven (vibrated). As a result, pulsed ultrasonic waves are output from the ultrasonic vibrator 48.

Further, in a case where each ultrasonic vibrator 48 receives reflected waves of ultrasonic wave (echoes) or the like, the ultrasonic vibrator 48 vibrates (is driven) in accordance with the reflected waves, and the piezoelectric element of each ultrasonic vibrator 48 generates an electrical signal. The electric signal is output as a reception signal from each ultrasonic vibrator 48 toward the ultrasonic processor device 14.

As described above, the ultrasonic vibrator unit 46 of the present embodiment is a convex type. In other words, in this embodiment, the N ultrasonic vibrators 48 included in the ultrasonic vibrator unit 46 are sequentially driven by an electronic switch such as a multiplexer 140, so that the ultrasonic waves are scanned within a scanning range along a curved surface on which the ultrasonic vibrator array 50 is disposed, for example, a range of about several tens of millimeters from the center of curvature of the curved surface.

As shown in FIG. 3, the backing material layer 54 supports the ultrasonic vibrator array 50 from the back side (the side opposite to the acoustic matching layer 76). Further, the backing material layer 54 has a function of attenuating ultrasonic waves propagated toward the back side of the ultrasonic vibrator array 50 among the ultrasonic waves emitted from the ultrasonic vibrator 48 or the ultrasonic waves (echoes) reflected from the observation target portion. A backing material is made of a material having rigidity such as hard rubber, in which an appropriate amount of an ultrasonic attenuating material (such as ferrite and ceramics) is added.

The acoustic matching layer 76 is provided to achieve acoustic impedance matching between the patient's body and a drive target vibrator. The acoustic matching layer 76 is disposed outside the ultrasonic vibrator array 50 (that is, the plurality of ultrasonic vibrators 48), and strictly speaking, is superimposed on the ultrasonic vibrator array 50 as shown in FIG. 3. By providing the acoustic matching layer 76, it is possible to increase transmittance of ultrasonic waves. As a material of the acoustic matching layer 76, various organic materials of which an acoustic impedance value is closer to that of the patient's body compared with the piezoelectric element of the ultrasonic vibrator 48 may be used. As the material of the acoustic matching layer 76, specifically, epoxy resin, silicone rubber, polyimide, polyethylene, and the like may be used.

The acoustic lens 78 is provided to converge ultrasonic waves emitted from the drive target vibrator toward the observation target portion, and is superimposed on the acoustic matching layer 76 as shown in FIG. 3. The acoustic lens 78 is made of, for example, a silicone resin (millable silicone rubber (HTV rubber), liquid silicone rubber (RTV rubber), or the like), a butadiene resin, a polyurethane resin, or the like, and powder of titanium oxide, alumina, silica, or the like may be mixed as necessary.

The FPC 60 is electrically connected to the electrodes provided in each ultrasonic vibrator 48. As shown in FIG. 3, each of the plurality of coaxial cables 56 is wired to the FPC 60 at one end thereof. In a case where the ultrasonic endoscope 12 is connected to the ultrasonic processor device 14 through the ultrasound connector 32a, each coaxial cable 56 is electrically connected to the ultrasonic processor device 14 at the other end thereof (on the side opposite to the FPC 60).

Endoscope Observation Part

The endoscope observation part 38 is a part provided for acquiring an endoscope image, and is disposed on a base end side with reference to the ultrasound observation part 36, in the distal end part 40 of the insertion part 22, as shown in FIGS. 2 and 3. As shown in FIGS. 2 and 3, the endoscope observation part 38 includes the observation window 82, an objective lens 84, the imaging element 86, the illumination window 88, the cleaning nozzle 90, a wiring cable 92, and the like.

As shown in FIG. 3, the observation window 82 is provided in a state of being inclined with respect to the axial direction (longitudinal axis direction of the insertion part 22), in the distal end part 40 of the insertion part 22. Light that is incident through the observation window 82 and is reflected by the observation target adjacent portion is imaged on an imaging surface of the imaging element 86 by the objective lens 84.

The imaging element 86 photoelectrically converts reflected light from the observation target adjacent portion that has passed through the observation window 82 and the objective lens 84 and is imaged on the imaging surface, and outputs an imaging signal. As the imaging element 86, a charge coupled device (CCD), a complementary metal oxide semiconductor (CMOS), or the like may be used. A captured image signal output by the imaging element 86 is transmitted to the endoscope processor device 16 by the universal cord 26 through the wiring cable 92 that elongates from the insertion part 22 to the operation part 24.

As shown in FIG. 2, the illumination window 88 is provided on both sides of the observation window 82. An emission end of a light guide (not shown) is connected to the illumination window 88. The light guide elongates from the insertion part 22 to the operation part 24, and an incident end thereof is connected to the light source device 18 connected through the universal cord 26. Illumination light emitted from the light source device 18 travels through the light guide, and is irradiated from the illumination window 88 toward the observation target adjacent portion.

Configuration of Ultrasonic Processor Device

As shown in FIG. 4, the ultrasonic processor device 14 includes the multiplexer 140, a reception circuit 142, a transmission circuit 144, an A/D converter 146, an image processing section 148, the system controller 152, and a display controller 154.

The reception circuit 142 and the transmission circuit 144 are electrically connected to the ultrasonic vibrator array 50 of the ultrasonic endoscope 12 through the multiplexer 140. The multiplexer 140 selects one or a plurality of ultrasonic vibrators 48 among N ultrasonic vibrators 48, and opens channels thereof.

The transmission circuit 144 is a circuit that supplies a drive voltage for ultrasonic transmission to the ultrasonic vibrator 48 selected by the multiplexer 140 in order to transmit ultrasonic waves from the ultrasonic vibrator unit 46. The drive voltage is a pulsed voltage signal, and is applied to the electrodes of the ultrasonic vibrator 48 to be driven through the universal cord 26 and the coaxial cable 56.

The reception circuit 142 is a circuit that receives an electrical signal output from the ultrasonic vibrator 48 that has received ultrasonic waves (echoes), that is, a reception signal. Further, the reception circuit 142 amplifies the reception signal received from the ultrasonic vibrator 48 in accordance with a control signal sent from the system controller 152, and delivers the amplified signal to the A/D converter 146. As shown in FIG. 4, the A/D converter 146 is connected to the reception circuit 142, converts a reception signal received from the reception circuit 142 from an analog signal to a digital signal, and outputs the converted digital signal to the image processing section 148.

The image processing section 148 is connected to the A/D converter 146 as shown in FIG. 4, and generates an ultrasound image based on a digital reception signal.

As shown in FIG. 4, the display controller 154 is connected to the image processing section 148, converts a signal of an ultrasound image generated by the image processing section 148 into an image signal based on a scan method of a normal television signal (raster conversion), performs a variety of necessary image processing such as gradation processing on the image signal, and outputs the image signal to the monitor 20.

The system controller 152 controls each section of the ultrasonic processor device 14, and is connected to the reception circuit 142, the transmission circuit 144, the A/D converter 146, and the image processing section 148 as shown in FIG. 4 to control these devices. As shown in FIG. 4, the system controller 152 is connected to the console 100, and controls each section of the ultrasonic processor device 14 in accordance with inspection information and control parameters input from the console 100 in inspecting a subject. Thus, an ultrasound image corresponding to an ultrasound image generation mode designated by the operator is acquired, and in particular, in the live mode, the ultrasound image is acquired at a constant frame rate as needed.

The system controller 152 includes various processors that execute processing by executing a program, a random access memory (RAM), and a read only memory (ROM).

The variety of processors in this specification may include a central processing unit (CPU) that is a general-purpose processor that executes a program to perform a variety of processing, a programmable logic device (PLD) that is a processor of which a circuit configuration is changeable after manufacturing, such as a field programmable gate array (FPGA), a dedicated electric circuit that is a processor having a circuit configuration that is dedicatedly designed for executing a specific process, such as an application specific integrated circuit (ASIC), or the like. More specifically, the structures of these various processors are electric circuits in which circuit elements such as semiconductor elements are combined.

The system controller 152 may be configured by one of various processors, or may be configured by a combination of two or more processors of the same type or different types (for example, a combination of a plurality of FPGAs or a combination of a CPU and an FPGA).

The system controller 152 performs the above-described failure diagnosis process at an unspecified timing in a period during which the ultrasonic endoscope 12 is not used in a state where the ultrasonic endoscope 12 is connected to the main body.

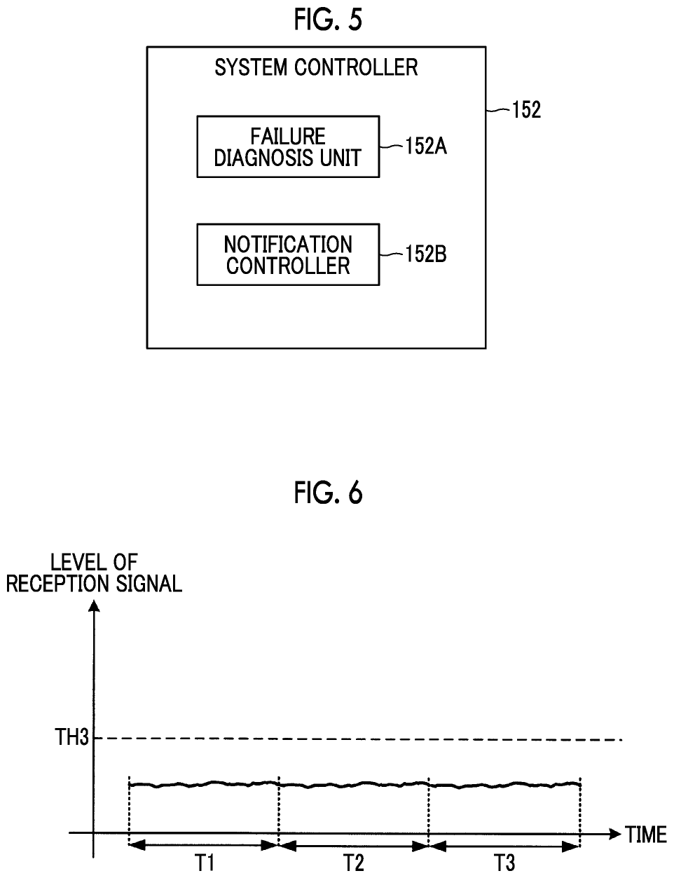

FIG. 5 is a diagram illustrating functional blocks of the system controller 152. A processor of the system controller 152 functions as a failure diagnosis unit 152A and a notification controller 152B by executing a failure diagnosis program of the ultrasonic endoscope apparatus. A failure diagnosis process is executed by these functional blocks. In this embodiment, the system controller 152 configures a failure diagnosis system of the ultrasonic endoscope apparatus.

The failure diagnosis unit 152A performs a process of controlling each of the N ultrasonic vibrators 48 so as not to transmit ultrasonic waves, selecting the N ultrasonic vibrators 48 one by one, and acquiring a reception signal of the selected ultrasonic vibrator 48. In this process, among a period during which each ultrasonic vibrator 48 is driven in a control sequence of the ultrasonic vibrator unit 46 in a case where an ultrasound image corresponding to one frame is acquired in a live mode or the like, a period during which a reception signal thereafter is output, the former period is replaced with a period during which each ultrasonic vibrator 48 is not driven. Further, in this process, in a period obtained by combining the period during which each ultrasonic vibrator 48 is not driven and a subsequent output period, a reception signal output from the ultrasonic vibrator 48 is acquired. With this process, reception signals at the time when ultrasonic waves are not transmitted are sequentially acquired from the respective N ultrasonic vibrators 48. The failure diagnosis unit 152A performs failure diagnosis of the ultrasonic endoscope apparatus 10 on the basis of the N reception signals acquired in this way.

The failure of the ultrasonic endoscope apparatus 10 refers to a state where noise mixed in a reception signal caused by various factors such as an abnormality of a device included in the ultrasonic endoscope 12 or an abnormality of a device such as a power source in the main body of the ultrasonic endoscope apparatus 10 is increased.

FIG. 6 is a diagram showing an example of a reception signal acquired in a case where ultrasonic waves are not transmitted. As shown in FIG. 6, the failure diagnosis unit 152A performs the above-described process, so that reception signals are acquired in the order of a period T1, a period T2, a period T3, and so on. The length of a period during which each reception signal is output is the same as a length obtained by combining the period during which each ultrasonic vibrator 48 is driven in the control sequence for generating an ultrasound image and the period during which the reception signal thereafter is output. In a case where no failure occurs in the ultrasonic endoscope apparatus 10, as shown in FIG. 6, each of the N reception signals is in a stable state at a low level.

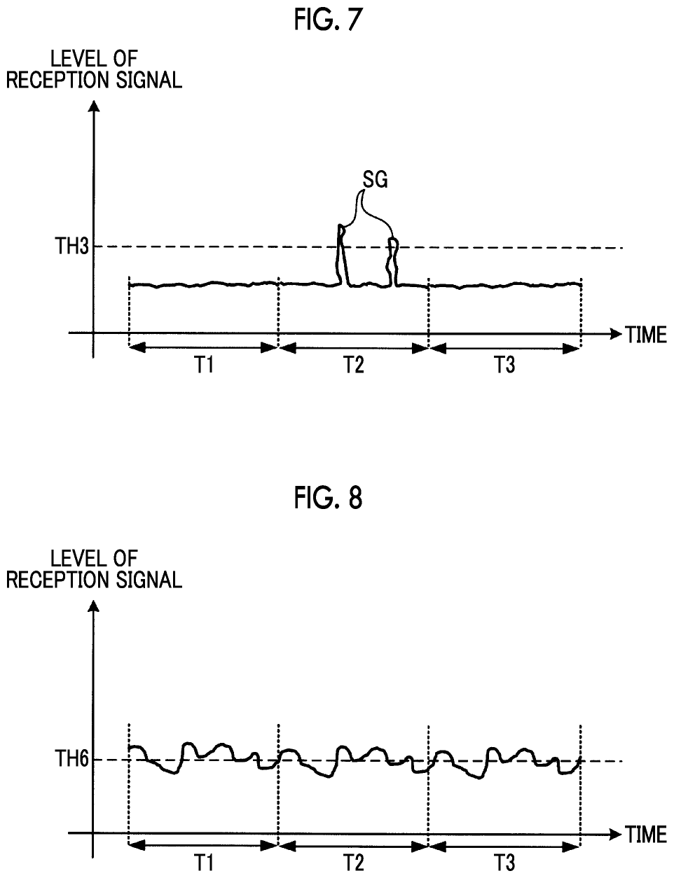

However, in a case where a failure occurs in the ultrasonic endoscope apparatus 10, as shown in FIG. 7, a state where a noise signal SG of a level that exceeds a predetermined threshold value TH3 is included in a reception signal frequently occurs.

The failure diagnosis unit 152A determines whether or not each of the N reception signals acquired in a state where ultrasonic waves are not transmitted includes the noise signal SG that exceeds the threshold value TH3, and sets the number of reception signals for which it is determined that the noise signal SG is included as an abnormality occurrence number X. It is preferable that the threshold value TH3 is not common to all the ultrasonic endoscopes 12 connectable to the main body and is individually determined for each ultrasonic endoscope 12.

Further, the failure diagnosis unit 152A diagnoses that there is a possibility of failure of the ultrasonic endoscope apparatus 10, in a case where the abnormality occurrence number X, an abnormality occurrence rate that is a ratio of the abnormality occurrence number X to the total number N of acquired reception signals, or an abnormality non-occurrence rate that is a ratio of (N−X) in N satisfies a predetermined condition.

In the ultrasonic endoscope apparatus 10, even in a case where noise is mixed in a reception signal of the ultrasonic vibrator 48, noise correction for correcting the noise may be performed in generating an ultrasound image. For example, an abnormality occurrence number or an abnormality occurrence rate in a case where the quality of the ultrasound image cannot be ensured by the above-described noise correction is set as a threshold value TH4. Further, the failure diagnosis unit 152A diagnoses that there is a possibility of failure of the ultrasonic endoscope apparatus 10 in a case where the abnormality occurrence number X or the abnormality occurrence rate is equal to or greater than the threshold value TH4. On the other hand, the failure diagnosis unit 152A diagnoses that there is no possibility of failure of the ultrasonic endoscope apparatus 10 in a case where the abnormality occurrence number X or the abnormality occurrence rate is smaller than the threshold value TH4.

Alternatively, a lower limit value of an abnormality non-occurrence rate in which the quality of the ultrasound image by the noise correction can be ensured is set as a threshold value TH5. Further, the failure diagnosis unit 152A diagnoses that there is a possibility of failure of the ultrasonic endoscope apparatus 10 in a case where the abnormality non-occurrence rate is smaller than the threshold value TH5. On the other hand, the failure diagnosis unit 152A diagnoses that there is no possibility of failure of the ultrasonic endoscope apparatus 10 in a case where the abnormality non-occurrence rate is equal to or higher than the threshold value TH5.

The notification controller 152B shown in FIG. 5 performs a notification process based on a diagnosis result of the failure diagnosis unit 152A. For example, in a case where a diagnosis result indicating that there is a possibility of failure is obtained, the notification controller 152B causes the monitor 20 to display a message for prompting maintenance of the ultrasonic endoscope apparatus 10, to thereby notify the user of maintenance recommendation of the ultrasonic endoscope apparatus 10. Instead of displaying the message on the monitor 20, the notification controller 152B may output the message through a speaker (not shown) provided in the ultrasonic endoscope apparatus 10. Alternatively, the notification controller 152B may transmit the message to an external electronic device connected to the ultrasonic endoscope apparatus 10 to notify an administrator or the user of the ultrasonic endoscope apparatus 10 of the necessity of maintenance.

As described above, according to the ultrasonic endoscope apparatus 10, it is possible to determine the possibility of failure of the ultrasonic endoscope apparatus 10 on the basis of a reception signal obtained from the ultrasonic vibrator 48 in a state where ultrasonic waves are not transmitted from the ultrasonic vibrator 48. In this way, by using a reception signal obtained in a state where ultrasonic waves are not transmitted from the ultrasonic vibrator 48 for failure diagnosis, it is possible to accurately determine a state of noise mixed in the apparatus. Thus, it is possible to appropriately execute maintenance of the apparatus.

Further, according to the ultrasonic endoscope apparatus 10, the failure diagnosis process is performed in a period during which the ultrasonic endoscope 12 is not used. In a case where the ultrasonic endoscope 12 is inserted into a body cavity and is in use, noise from various devices such as an electric scalpel used at the time of inspection may be mixed into a reception signal. By performing the failure diagnosis process in a period during which the ultrasonic endoscope 12 is not used, it is possible to eliminate the said influence of noise, and to perform the failure diagnosis with high accuracy.

The failure diagnosis unit 152A acquires a reception signal from each of the N ultrasonic vibrators 48 in a state where ultrasonic waves are not transmitted, but the invention is not limited thereto. The failure diagnosis unit 152A may acquire reception signals from at least two ultrasonic vibrators 48 among the N ultrasonic vibrators 48 in a state where ultrasonic waves are not transmitted, and may determine an abnormality based on the acquired reception signals. Even in this case, it is possible to determine the presence or absence of failure in accordance with the magnitude of the abnormality occurrence number, the abnormality occurrence rate, or the abnormality non-occurrence rate.

Modification Example of Ultrasonic Endoscope Apparatus

The functional blocks of the system controller 152 in the ultrasonic endoscope apparatus 10 of a first modification example are the same as those in FIG. 5, but the functions of the failure diagnosis unit 152A are partially different. In this modification example, similarly, the system controller 152 configures the failure diagnosis system of the ultrasonic endoscope apparatus.

This modification example is the same as the above-described embodiment in that the failure diagnosis unit 152A performs failure diagnosis of the ultrasonic endoscope apparatus 10 based on N reception signals in a state where ultrasonic waves are not transmitted, acquired as described above, but its diagnosis method is different.

FIG. 8 is a diagram showing an example of a reception signal acquired in a case where ultrasonic waves are not transmitted. Depending on the cause of abnormality of the ultrasonic endoscope apparatus 10, there is a case where noise is superimposed on a reception signal as a whole and an average level of the respective reception signals is high compared with the state shown in FIG. 6, as shown in FIG. 8. Further, in a case where the average level becomes excessively high (for example, reaches a predetermined threshold value TH6), there is a possibility that the quality of an ultrasound image may not be maintained. Thus, the failure diagnosis unit 152A in the modification example calculates an average level of the N reception signals, diagnoses that there is a possibility of failure of the ultrasonic endoscope apparatus 10 in a case where the average level is equal to or higher than the threshold value TH6, and diagnoses that there is no possibility of failure of the ultrasonic endoscope apparatus 10 in a case where the average level is smaller than the threshold value TH6.

Alternatively, the failure diagnosis unit 152A calculates the average level of the respective N reception signals, and calculates the number of reception signals of which the average level is equal to or higher than the threshold value TH6 as an abnormality occurrence number. Further, in a case where the abnormality occurrence number or the abnormality occurrence rate that is the ratio of the abnormality occurrence number to N is equal to or higher than the threshold value TH4, the failure diagnosis unit 152A may diagnose that there is a possibility of failure of the ultrasonic endoscope apparatus 10, and in a case where the abnormality occurrence number or the abnormality occurrence rate is smaller than the threshold value TH4, the failure diagnosis unit 152A may diagnose that there is no possibility of failure of the ultrasonic endoscope apparatus 10.

Alternatively, the failure diagnosis unit 152A may calculate an abnormality non-occurrence rate that is a ratio of (N-abnormality occurrence number) to N, may diagnose that there is a possibility of failure of the ultrasonic endoscope apparatus 10 in a case where the abnormality non-occurrence rate is smaller than the threshold value TH5, and may diagnose that there is no possibility of failure of the ultrasonic endoscope apparatus 10 in a case where the abnormality non-occurrence rate is equal to or higher than the threshold value TH5.

As described above, according to the ultrasonic endoscope apparatus 10 of the modification example, it is possible to determine the possibility of failure of the ultrasonic endoscope apparatus 10 on the basis of a reception signal obtained from the ultrasonic vibrator 48 in a state where ultrasonic waves are not transmitted from the ultrasonic vibrator 48. In this way, by using a reception signal obtained in a state where ultrasonic waves are not transmitted from the ultrasonic vibrator 48 for failure diagnosis, it is possible to accurately determine a state of noise mixed in the apparatus. Thus, it is possible to appropriately execute maintenance of the apparatus.

In this modification example, the failure diagnosis unit 152A acquires a reception signal from each of the N ultrasonic vibrators 48 in a state where ultrasonic waves are not transmitted, but the invention is not limited thereto. The failure diagnosis unit 152A may acquire a reception signal from at least one ultrasonic vibrator 48 among the N ultrasonic vibrators 48 in a state where ultrasonic waves are not transmitted, and may determine the presence or absence of failure, on the basis of the magnitude of an average level of all the acquired reception signals, the number of reception signals of which the average level exceeds the threshold value TH6, or the like.

The respective functional blocks of the system controller 152 in the above-described embodiment and its modification example may be configured to be provided in a processor included in the endoscope processor device 16, or may be configured to be provided in a processor included in an external device such as an external server connectable to the ultrasonic endoscope apparatus 10. In the former configuration, the processor of the endoscope processor device 16 forms the failure diagnosis system. In the latter configuration, the processor of the external device forms the failure diagnosis system.

As described above, the following content is disclosed in this specification.

(1) A failure diagnosis system of an ultrasonic endoscope apparatus comprising a failure diagnosis unit that acquires a reception signal of an ultrasonic vibrator of an ultrasonic endoscope in a state where ultrasonic waves are not transmitted from the ultrasonic vibrator and performs failure diagnosis of the ultrasonic endoscope apparatus including the ultrasonic endoscope on the basis of the reception signal.

(2) The failure diagnosis system of the ultrasonic endoscope apparatus according to (1), wherein the failure diagnosis unit acquires the reception signal of each of a plurality of the ultrasonic vibrators included in the ultrasonic endoscope, and performs the failure diagnosis on the basis of the number of the reception signals including a signal having a value exceeding a predetermined value.

(3) The failure diagnosis system of the ultrasonic endoscope apparatus according to (1), wherein the failure diagnosis unit acquires the reception signal of each of a plurality of the ultrasonic vibrators included in the ultrasonic endoscope, and performs the failure diagnosis on the basis of the number of the reception signals having an average level exceeding a predetermined value.

(4) The failure diagnosis system of the ultrasonic endoscope apparatus according to (2) or (3), wherein the failure diagnosis unit diagnoses that there is a possibility of failure of the ultrasonic endoscope apparatus, in a case where the number, a ratio of the number in a total number of the acquired reception signals, or a ratio of the number obtained by subtracting the number from the total number in the total number satisfies a predetermined condition.

(5) The failure diagnosis system of the ultrasonic endoscope apparatus according to (1), wherein the failure diagnosis unit acquires the reception signal of each of a plurality of the ultrasonic vibrators included in the ultrasonic endoscope, and performs the failure diagnosis on the basis of on an average level of all the acquired reception signals.

(6) The failure diagnosis system of the ultrasonic endoscope apparatus according to (5), wherein the failure diagnosis unit diagnoses that there is a possibility of failure of the ultrasonic endoscope apparatus, in a case where the average level is equal to or higher than a predetermined value.

(7) The failure diagnosis system of the ultrasonic endoscope apparatus according to any one of (1) to (6), wherein the failure diagnosis unit performs the failure diagnosis in a period during which the ultrasonic endoscope is not used.

(8) The failure diagnosis system of the ultrasonic endoscope apparatus according to any one of (1) to (7), further comprising: a notification controller that performs a notification process on the basis of a diagnosis result, in a case where it is diagnosed that there is a possibility of failure by the failure diagnosis unit.

(9) The failure diagnosis system of the ultrasonic endoscope apparatus according to any one of (1) to (8), wherein the failure diagnosis unit is provided in a main body of the ultrasonic endoscope apparatus.

(10) A failure diagnosis method of an ultrasonic endoscope apparatus comprising: acquiring a reception signal of an ultrasonic vibrator of an ultrasonic endoscope in a state where ultrasonic waves are not transmitted from the ultrasonic vibrator; and performing failure diagnosis of the ultrasonic endoscope apparatus including the ultrasonic endoscope on the basis of the reception signal.

(11) A non-transitory computer readable recording medium storing a failure diagnosis program of an ultrasonic endoscope apparatus for causing a computer to execute: a step of acquiring a reception signal of an ultrasonic vibrator of an ultrasonic endoscope in a state where ultrasonic waves are not transmitted from the ultrasonic vibrator; and a step of performing failure diagnosis of the ultrasonic endoscope apparatus including the ultrasonic endoscope on the basis of the reception signal.

### EXPLANATION OF REFERENCES

10: ultrasonic endoscope apparatus
12: ultrasonic endoscope
14: ultrasonic processor device
16: endoscope processor device
18: light source device
20: monitor
21a: water supply tank
21b: suction pump
21c: air supply pump
22: insertion part
24: operation part
26: universal cord
28a: air/water supply button
28b: suction button
30: treatment instrument insertion port
32a: ultrasound connector
32b: endoscope connector
32c: light source connector
36: ultrasound observation part
37: balloon
38: endoscope observation part
40: distal end part
42: bending part
43: flexible part
44: treatment instrument outlet
45: treatment instrument channel
46: ultrasonic vibrator unit
47: water supply port
48: ultrasonic vibrator
50: ultrasonic vibrator array
54: backing material layer
56: coaxial cable
60: FPC
76: acoustic matching layer
78: acoustic lens
82: observation window
84: objective lens
86: imaging element
88: illumination window
100: console
140: multiplexer
142: reception circuit
144: transmission circuit
146: A/D converter
148: image processing section
152: system controller
152A: failure diagnosis unit
152B: notification controller
SG: noise signal

What is claimed is:

1. A failure diagnosis system of an ultrasonic endoscope apparatus comprising:
a failure diagnosis unit that acquires a reception signal of an ultrasonic vibrator of an ultrasonic endoscope in a state where ultrasonic waves are not transmitted from the ultrasonic vibrator and performs failure diagnosis of the ultrasonic endoscope apparatus including the ultrasonic endoscope on the basis of the reception signal.

2. The failure diagnosis system of the ultrasonic endoscope apparatus according to claim 1,
wherein the failure diagnosis unit acquires the reception signal of each of a plurality of the ultrasonic vibrators included in the ultrasonic endoscope, and performs the failure diagnosis on the basis of the number of the reception signals including a signal having a value exceeding a predetermined value.

3. The failure diagnosis system of the ultrasonic endoscope apparatus according to claim 1,
wherein the failure diagnosis unit acquires the reception signal of each of a plurality of the ultrasonic vibrators included in the ultrasonic endoscope, and performs the failure diagnosis on the basis of the number of the reception signals of which an average level exceeds a predetermined value.

4. The failure diagnosis system of the ultrasonic endoscope apparatus according to claim 2,
wherein the failure diagnosis unit diagnoses that there is a possibility of failure of the ultrasonic endoscope apparatus, in a case where the number, a ratio of the number in a total number of the acquired reception signals, or a ratio of the number obtained by subtracting the number from the total number in the total number satisfies a predetermined condition.

5. The failure diagnosis system of the ultrasonic endoscope apparatus according to claim 3,
wherein the failure diagnosis unit diagnoses that there is a possibility of failure of the ultrasonic endoscope apparatus, in a case where the number, a ratio of the number in a total number of the acquired reception signals, or a ratio of the number obtained by subtracting the number from the total number in the total number satisfies a predetermined condition.

6. The failure diagnosis system of the ultrasonic endoscope apparatus according to claim 1,
wherein the failure diagnosis unit acquires the reception signal of each of a plurality of the ultrasonic vibrators included in the ultrasonic endoscope, and performs the failure diagnosis on the basis of an average level of all the acquired reception signals.

7. The failure diagnosis system of the ultrasonic endoscope apparatus according to claim 6,
wherein the failure diagnosis unit diagnoses that there is a possibility of failure of the ultrasonic endoscope apparatus, in a case where the average level is equal to or higher than a predetermined value.

8. The failure diagnosis system of the ultrasonic endoscope apparatus according to claim 1,
wherein the failure diagnosis unit performs the failure diagnosis in a period during which the ultrasonic endoscope is not used.

9. The failure diagnosis system of the ultrasonic endoscope apparatus according to claim 2,
wherein the failure diagnosis unit performs the failure diagnosis in a period during which the ultrasonic endoscope is not used.

10. The failure diagnosis system of the ultrasonic endoscope apparatus according to claim 3,
wherein the failure diagnosis unit performs the failure diagnosis in a period during which the ultrasonic endoscope is not used.

11. The failure diagnosis system of the ultrasonic endoscope apparatus according to claim 4,
wherein the failure diagnosis unit performs the failure diagnosis in a period during which the ultrasonic endoscope is not used.

12. The failure diagnosis system of the ultrasonic endoscope apparatus according to claim 5,
wherein the failure diagnosis unit performs the failure diagnosis in a period during which the ultrasonic endoscope is not used.

13. The failure diagnosis system of the ultrasonic endoscope apparatus according to claim 6,
wherein the failure diagnosis unit performs the failure diagnosis in a period during which the ultrasonic endoscope is not used.

14. The failure diagnosis system of the ultrasonic endoscope apparatus according to claim 7,
wherein the failure diagnosis unit performs the failure diagnosis in a period during which the ultrasonic endoscope is not used.

15. The failure diagnosis system of the ultrasonic endoscope apparatus according to claim 1, further comprising:
a notification controller that performs a notification process on the basis of a diagnosis result, in a case where it is diagnosed that there is a possibility of failure by the failure diagnosis unit.

16. The failure diagnosis system of the ultrasonic endoscope apparatus according to claim 2, further comprising:
a notification controller that performs a notification process on the basis of a diagnosis result, in a case where it is diagnosed that there is a possibility of failure by the failure diagnosis unit.

17. The failure diagnosis system of the ultrasonic endoscope apparatus according to claim 1,
wherein the failure diagnosis unit is provided in a main body of the ultrasonic endoscope apparatus.

18. A failure diagnosis method of the ultrasonic endoscope apparatus according to claim 1 comprising: acquiring a reception signal of the ultrasonic vibrator of the ultrasonic endoscope in a state where ultrasonic waves are not transmitted from the ultrasonic vibrator; and performing failure diagnosis of the ultrasonic endoscope apparatus including the ultrasonic endoscope on the basis of the reception signal.

19. A non-transitory computer readable recording medium storing a failure diagnosis program of an ultrasonic endoscope apparatus for causing a computer to execute: a step of acquiring a reception signal of an ultrasonic vibrator of an ultrasonic endoscope in a state where ultrasonic waves are not transmitted from the ultrasonic vibrator; and a step of performing failure diagnosis of the ultrasonic endoscope apparatus including the ultrasonic endoscope on the basis of the reception signal.

20. A failure diagnosis system of an ultrasonic endoscope apparatus comprising:
a processor configured to acquire a reception signal of an ultrasonic vibrator of an ultrasonic endoscope in a state where ultrasonic waves are not transmitted from the ultrasonic vibrator and perform failure diagnosis of the ultrasonic endoscope apparatus including the ultrasonic endoscope on the basis of the reception signal.
